(19) United States
(12) Patent Application Publication
(10) Pub. No.: US 2026/0325778 A1
(43) Pub. Date: Oct. 22, 2026

(54) ESOPHAGUS INTUBATION DEVICE

(71) Applicant: Quirino G. Orlandi, Edina, MN (US)

(72) Inventor: Quirino G. Orlandi, Edina, MN (US)

(21) Appl. No.: 19/654,335

(22) Filed: Apr. 21, 2026

Related U.S. Application Data
(60) Provisional application No. 63/792,776, filed on Apr. 22, 2025.

(51) Int. Cl. A61M 16/04

(52) U.S. Cl. CPC A61M 16/0493; A61M 16/0477; A61M 2205/0266; A61M 2205/3606

(57) ABSTRACT

A bite block can be configured to keep the mouth open during a medical procedure. The elongated tube can extend from the bite block. An elongated tube can be operable between a collapsed configuration for insertion through the mouth into a pharynx and into an esophagus of a patient and an expanded configuration. In the expanded configuration, the elongated tube can include a lumen extending from a proximal end of the elongated tube to a distal end of the elongated tube. The lumen can guide an intubation probe to the esophagus of the patient. An outer wall can surround the lumen and can define part of a fluid channel. The outer wall can include exits that can be fluidically coupled to the fluid channel to direct a fluid to portions of the anatomy of the patient or to draw debris from the portions of the anatomy of the patient.

### CLAIM OF PRIORITY

This patent application claims the benefit of priority, under 35 U.S.C. Section 119(e), to Dr. Quirino G. Orlandi, U.S. patent application Ser. No. 63/792,776, entitled “ESOPHAGUS INTUBATION DEVICE,” filed on Apr. 22, 2025, (Attorney Docket No. 6536.001PRV), which is hereby incorporated by reference herein in its entirety.

### TECHNICAL FIELD

Examples described herein generally relate to an intubation device and more specifically to an esophagus intubation device.

### BACKGROUND

Esophageal intubation is a medical procedure that facilitates access to the esophagus for diagnostic or therapeutic purposes, such as during endoscopic examinations or the placement of feeding tubes. The procedure can include the insertion of a flexible or rigid tube into the esophagus, typically through the oral cavity, to enable visualization, delivery of treatments, or management of esophageal conditions. Proper technique can help minimize risks such as mucosal injury, airway compromise, or patient discomfort.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates an example of an esophagus intubation device.

FIG. 2 illustrates an example of an esophagus intubation device.

FIG. 3 illustrates a portion of an example of an esophagus intubation device.

FIG. 4 illustrates a cross-sectional view of an example of the esophagus intubation device from FIG. 3 taken along indicator line 4-4.

FIG. 5 illustrates a portion of an example of an esophagus intubation device.

FIG. 6 illustrates a distal section of an example of an esophagus intubation device.

FIG. 7 illustrates a portion of an example of an esophagus intubation device including expansion of the esophagus intubation device.

FIG. 8 illustrates a schematic diagram of an example of an expandable bite block.

FIG. 9 illustrates a schematic diagram of an example of an expandable bite block.

FIG. 10 illustrates a schematic diagram of an example of an expandable bite block.

FIG. 11 illustrates an example of a method for using an esophagus intubation device.

### DETAILED DESCRIPTION

The process of esophageal intubation can be inherently challenging due to several factors. Firstly, the anatomy of the upper airway and esophagus presents a narrow window for precise tube placement, increasing the risk of misplacement into the trachea or other structures. This risk can be exacerbated by patient-specific factors such as anatomical variations, obesity, or conditions like obstructive sleep apnea that can complicate the intubation process. Additionally, esophageal intubation can require a high level of skill and experience to navigate the delicate balance between ensuring proper tube placement and avoiding complications such as tissue damage or aspiration.

The lack of immediate and accurate feedback during the procedure can make it difficult for practitioners to confirm whether the tube is correctly positioned in the esophagus. This uncertainty necessitates the use of devices that can cause less trauma to the walls of the mouth, pharynx, or esophagus and can be more easily inserted therein.

Overall, the complexity and potential risks associated with esophageal intubation highlight the need for innovative solutions that can simplify the process, improve accuracy, and enhance patient safety. The present disclosure is directed to a device that can address at least the above-mentioned problems with esophageal intubation.

In examples, a device can include a bite block and an elongated tube. The bite block can be configured to be inserted into the mouth of a patient to keep the mouth open during a medical procedure. The elongated tube can extend from the bite block. The elongated tube can be operable between a collapsed configuration for insertion into an esophagus of a patient and an expanded configuration. In the expanded configuration, the elongated tube can include a lumen extending from a proximal end of the elongated tube adjacent to the bite block to a distal end of the elongated tube. The lumen can be configured to guide an intubation probe, e.g., a transesophageal echocardiography (TEE) probe, or any other probe that can be inserted into the mouth, pharynx, or esophagus of the patient, into the proximal end of the elongated tube and out of the distal end of the elongated tube and into the esophagus. An outer wall can surround the lumen and can define part of a fluid channel. The outer wall can include one or more exits that can be fluidically coupled to the fluid channel to direct a fluid to one or more portions of the esophagus or to draw debris from the one or more portions of the esophagus.

In examples, a method of using an esophagus intubation device can include inserting an elongated tube of the esophagus intubation device within a mouth and into the esophagus of a patient. Positioning a bite block within the mouth of the patient, the bite block coupled to a proximal portion of the elongated tube. Delivering, via a channel of the elongated tube, a fluid to the esophagus of the patient. The method can also include inserting, within a lumen of the elongated tube, an intubation probe within the esophagus of the patient to perform the medical procedure.

The above discussion is intended to provide an overview of the subject matter of the present patent application. It is not intended to provide an exclusive or exhaustive explanation of the invention. The description below is included to provide further information about the present patent application.

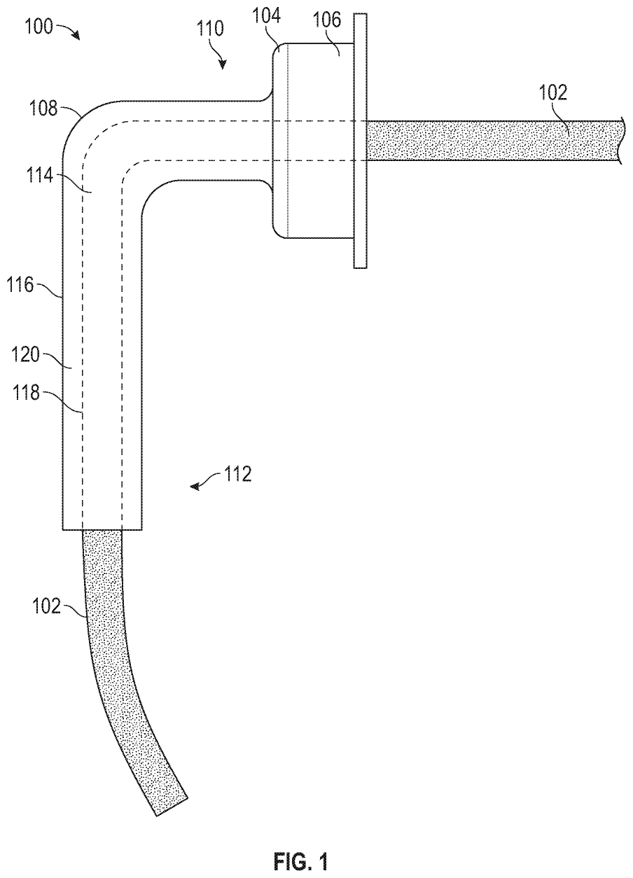

FIG. 1 illustrates an example of an esophagus intubation device 100. The esophagus intubation device 100 can be configured to guide an intubation probe 102 within the esophagus of a patient. The esophagus intubation device 100 can include a bite block 104 and an elongated tube 108.

The intubation probe 102 can be a transesophageal echocardiography probe, any other probe insertable through the mouth, pharynx, or esophagus of the patient, or the like. The intubation probe 102 can be used to capture images, biopsies, or deliver medication to the esophagus. The esophagus intubation device 100 can be configured to direct the intubation probe 102 into the esophagus. Moreover, the esophagus intubation device 100 can be configured to increase predictability and ease the insertion of the intubation probe 102 in the esophagus of the patient.

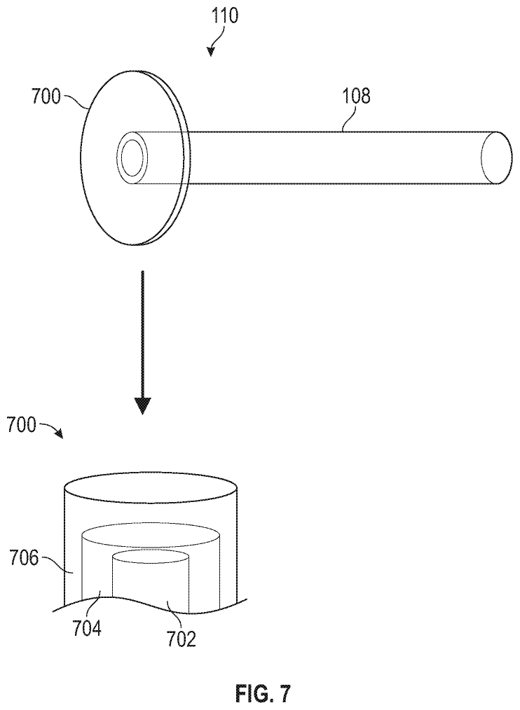

The bite block 104 can be inserted into the mouth of a patient to keep the mouth open during a medical procedure. The bite block 104 can also be configured to keep the tongue of the patient clear of the medical procedure. The bite block 104 can engage with oral members of the patient to hold the mouth of the patient open to provide access to the esophagus and other anatomy for medical professionals. The bite block 104 can define a cavity 106. The cavity 106 can provide the clinician access to the mouth of the patient or the esophagus of the patient through the bite block 104. Although in the present disclosure, the bite block 104 is shown as a part of the esophagus intubation device 100, the bite block 104 can be a standalone medical device or can be incorporated in other medical devices that can be used in or around the mouth of the patient. Different examples of the bite block 104 will be discussed with reference to FIGS. 7-10.

In examples, the bite block 104 can be configured to receive the elongated tube 108 to removably couple the bite block 104 to the elongated tube 108. The bite block 104 can be configured to be inserted into the mouth of the patient in a “low profile” state and expanded to an expanded state to spread the teeth of the patient apart, and open the mouth to provide clearance between the tongue and other tissue of the mouth to provide better access to the pharynx and ultimately, the esophagus of the patient. As discussed herein, the bite block 104 can be configured to move from the “low profile” state to the expanded state, or another intermediate state, after insertion into the mouth, or as the bite block 104 is being inserted into the mouth.

A proximal end 110 of the elongated tube 108 can extend from the bite block 104. The distal end 112 of the elongated tube 108 can be insertable through the mouth and into the esophagus of the patient. The elongated tube 108 can include a lumen 114 that can extend from the proximal end 110 to the distal end 112. The lumen 114 can be connected to the cavity 106. Thus, the intubation probe 102 can be inserted into the lumen 114 through the cavity 106. The lumen 114 can guide the intubation probe 102 out the distal end 112 of the elongated tube 108 and into the esophagus of the patient. In examples, the elongated tube 108 can include a length between 20 and 25 cm for adults or between 6 and 15 cm for children. In examples, the elongated tube 108 can include a length between 15 and 35 cm for adults and between 3 and 20 cm for children. The elongated tube 108, when in the expanded configuration, can include an outer diameter between 8 and 15 mm for adults and between 3 and 11 mm for children. In examples, the elongated tube 108, when in the expanded configuration, can include an outer diameter between 5 and 30 mm. The elongated tube 108, when in the contracted configuration, can include an outer diameter between 3 and 7 mm for adults and between 2 and 6 mm for children. In the contracted configuration, the outside diameter of the elongated tube 108 can be configured to be less than the outer dimension of the intubation probe 102. In examples, the elongated tube 108 can also be more flexible than the intubation probe 102 to help facilitate insertion of the elongated tube 108 into the mouth, pharynx, or esophagus of the patient.

The elongated tube 108 can include an outer wall 116 and an inner wall 118. A channel 120 can be located between the inner wall 118 and the outer wall 116. The lumen 114 can be located within or defined by the inner wall 118. The channel 120 can be configured to provide fluid (e.g., saline, water, lidocaine, glucose, any biocompatible lubricant, combinations thereof, or the like) to the esophagus or can be configured to provide suction to the esophagus intubation device 100. In some examples, the channel 120 can be configured to provide fluid suction to the esophagus. The fluid introduced via the channel 120 can help the clinician prepare the patient for the medical procedure, assist the clinician during the medical procedure, or can help with the recovery of the patient after the medical procedure. In examples, a cooling fluid can be delivered via the channel 120 to draw heat away from the intubation probe 102 to help reduce the likelihood of the intubation probe 102 overheating during longer medical procedures. In other examples, the channel 120 can include a heat sink to draw heat away from the intubation probe 102 or the channel 120 can be actively cooled via any cooling means that can be used within the elongated tube 108 to help maintain the temperature of the intubation probe 102 throughout medical procedures. The suction introduced via the channel 120 can help the clinician remove debris (e.g., tissue, saliva, blood, other surgical debris, or the like) from the esophagus of the patient before, during, or after the medical procedure. In examples, the channel 120 can have a diameter between 0.5 and 3 mm.

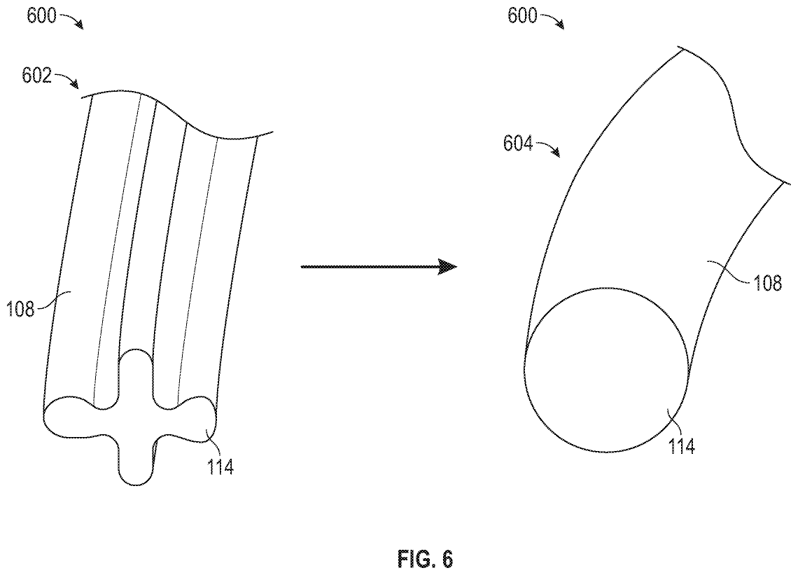

As will be discussed herein in more detail, the esophagus intubation device 100 can be configured to be inserted through a mouth of a patient and into the esophagus of the patient. The elongated tube 108 can be operable between a collapsed configuration (e.g., the collapsed configuration 602, FIG. 6) for insertion into the esophagus of a patient and an expanded configuration (e.g., the expanded configuration 604, FIG. 6) to enable the clinician to perform the medical procedure. The expanded configuration can define the lumen 114, the channel 120, and other features of the elongated tube 108.

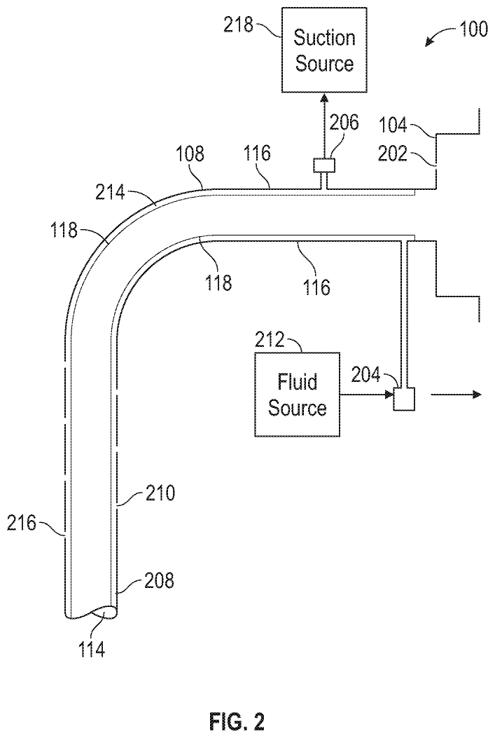

FIG. 2 illustrates an example of an esophagus intubation device 100. The esophagus intubation device 100 can include an aperture 202 and a first side port 204 and a second side port 206. The esophagus intubation device 100 can include a fluid channel 208 and a suction channel 214. The fluid channel 208 can be fluidly connected to a fluid source 212 by the first side port 204. The suction channel 214 can be fluidly connected to the suction source 218 by the second side port 206. The fluid channel 208 and the suction channel 214 can be fluidically isolated from one another to permit independent control of the fluid supplied through the fluid channel 208 and the suction through the suction channel 214. The fluid channel 208 and the suction channel 214 can also be implemented as a single channel, for example, as the channel 120 (FIG. 1). In examples, the first side port 204 and the second side port 206 can be adjacent to the bite block 104. In examples, the first side port 204 and the second side port 206 can extend from the elongated tube 108 proximally, such that the first side port 204 and the second side port 206 can be connected to the fluid source 212 and the suction inlets 216 outside the mouth of the patient.

The aperture 202 can be configured to improve airflow to permit breathing while the esophagus intubation device 100 is inserted into the esophagus of the patient. The aperture 202 can also be used to clear debris or liquid within or around the mouth of the patient. The aperture 202 can fluidly connect the mouth of the patient to the cavity 106. Thus, the aperture 202 can permit a patient to breathe more easily during the medical procedure as compared to a bite block that does not include the aperture 202. In examples, the aperture 202 can include a filter or other device to prevent unintended debris or liquid from escaping the mouth of the patient into the cavity 106. In examples, the filter can also prevent fluid within the cavity 106 from entering the mouth of the patient through the aperture 202.

The first side port 204 can couple to an opening in the outer wall 116 and be configured to fluidly connect a fluid source 212 and the fluid channel 208. In examples, the fluid source 212 can be configured to deliver more than one fluid to the fluid channel 208 through the first side port 204. For example, the fluid source 212 can be configured to provide saline, water, lidocaine, glucose, any biocompatible lubricant, combinations thereof, or the like, to the fluid channel 208 through the first side port 204. The first side port 204 can extend laterally away from the elongated tube 108. The lateral extension of the first side port 204 can move the connections to the fluid source 212 farther away from the mouth of the patient to provide more room for the clinician to work in and around the mouth.

The second side port 206 can couple to an opening in the outer wall 116 and be configured to connect a suction source 218 and the suction channel 214. In examples, the suction source 218 can be configured to provide suction to the suction channel 214 through the second side port 206. For example, the suction source 218 can be configured to provide suction to draw tissue, saliva, blood, other surgical debris, combinations thereof, or the like, away from the surgical site and into the suction channel 214. In examples, a debris collection receptacle can be included between the second side port 206 and the suction source 218. The debris collection receptacle can also be included between the suction channel 214 and the second side port 206. The second side port 206 can extend laterally away from the elongated tube 108. The lateral extension of the second side port 206 can move the connections to the suction source 218 farther away from the mouth of the patient to provide more room for the clinician to work in and around the mouth.

The fluid channel 208 can include fluid exits 210 and the suction channel 214 can include suction inlets 216. The fluid exits 210 can extend through the outer wall 116 to fluidly connect the fluid channel 208 to the esophagus to provide fluid from the fluid source 212 to the esophagus. The suction inlets 216 can extend through the outer wall 116 to draw debris from within the esophagus into the suction channel 214 and toward the suction source 218.

The fluid exits 210 and the suction inlets 216 can be axially spaced along the outer wall 116 of the elongated tube 108. As the fluid exits 210 and the suction inlets 216 extend axially along the outer wall 116, the fluid exits 210 can be circumferentially offset from the suction inlets 216. The circumferential offset between the fluid exits 210 and the suction inlets 216 can ensure that the fluid can be presented into the esophagus of the patient and debris can be removed from the esophagus of the patient simultaneously. As the fluid exits 210 extend axially along the outer wall 116, adjacent fluid exits of the fluid exits 210 can be circumferentially offset from each other. The circumferential offset between adjacent fluid exits of the fluid exits 210 can help distribute the fluid to multiple portions of the esophagus. As the suction inlets 216 extend axially along the outer wall 116, adjacent suction inlets of the suction inlets 216 can be circumferentially offset from each other. The circumferential offset of the suction inlets 216 can help distribute the suction force provided by the suction source 218 to multiple portions of the esophagus.

The fluid exits 210 can couple to an opening in the outer wall 116 and into the fluid channel 208. The fluid exits 210 can be spaced along the outer wall 116 to provide fluid along one or more portions of the esophagus. As the fluid exits 210 extend along the outer wall 116 from the bite block 104 to the distal end 112, a diameter of the fluid exits 210 can increase. Having the diameter of the fluid exits 210 increase as the fluid exits 210 extend along the outer wall 116 from the bite block 104 to the distal end 112 can evenly distribute the fluid to the different portions of the esophagus. In some examples, the diameter of the fluid exits 210 can be substantially equal as they extend along the outer wall 116. The fluid channel 208 and the first side port 204 can be configured to fluidly connect the fluid exits 210 to the fluid source 212 to provide the fluid along one or more portions of the esophagus. In examples, the fluid exits 210 can have a diameter between 0.5 and 10 mm. In examples, the flow of the fluid from the fluid source 212 into the fluid channel 208 and out the fluid exits 210 can be metered by a pump, a rate of dispensing from a syringe or other medical device, or the like.

As shown in FIG. 2, the fluid exits 210 can include a circular profile. In examples, the fluid exits 210 can include an oval, oblong, slit-like, rectangular, or the like profile to fluidly connect the fluid channel 208 to the inner walls of the mouth, pharynx, or esophagus of the patient. The major dimension of the fluid exits 210 can be variable as the fluid exits 210 extend along the outer wall 116 from the bite block 104 to the distal end 112 to distribute fluid throughout the mouth, pharynx, and esophagus of the patient. In examples, the variable dimension of the fluid exits 210 can facilitate delivery of the fluid from the fluid source 212 to areas of the mouth, pharynx, or esophagus with variable constraints, e.g., the internal diameter of the mouth, pharynx, or esophagus that can engage the fluid exits 210 to restrict or limit the distribution of the fluid. In examples, the variable dimension of the fluid exits 210 can be between 0.5 and 10 mm.

The suction inlets 216 can couple to an opening in the outer wall 116 and into the suction channel 214. The suction inlets 216 can be configured to fluidly connect the suction inlets 216 to the suction source 218 to provide the suction along one or more portions of the esophagus. The suction inlets 216 can be spaced along the outer wall 116 to provide suction along one or more portions of the esophagus. As the suction inlets 216 extend along the outer wall 116, from the bite block 104 to the distal end 112, a diameter of the suction inlets 216 can increase. Having the diameter of the suction inlets 216 increase as the suction inlets 216 extend along the outer wall 116, from the bite block 104 to the distal end 112, can evenly distribute a suction force to the different portions of the esophagus. In examples, the suction inlets 216 can have a diameter between 0.5 and 10 mm. In examples, the suction inlets 216 can have a smaller diameter than the fluid exits 210 to encourage the fluid from the fluid source 212 to flow into the esophagus of the patient. In other examples, the suction force can be controlled with a pump, and a controller can control the pumping rate of the fluid from the fluid source 212 relative to the suction force from the suction source 218 to control an amount of fluid dispensed from the elongated tube 108 to the mouth, pharynx, and esophagus of the patient.

As shown in FIG. 2, the suction inlets 216 can include a circular profile. In examples, the suction inlets 216 can include an oval, oblong, slit-like, rectangular, or the like profile to fluidly connect the suction channel 214 to the inner walls of the mouth, pharynx, or esophagus of the patient. The major dimension of the suction inlets 216 can be variable as the suction inlets 216 extend along the outer wall 116 from the bite block 104 to the distal end 112 to provide suction force throughout the mouth, pharynx, and esophagus of the patient. In examples, the variable dimension of the suction inlets 216 can facilitate suction force to areas of the mouth, pharynx, or esophagus with variable constraints, e.g., the internal diameter of the mouth, pharynx, or esophagus that can engage the suction inlets 216 to restrict or limit the suction into the suction channel 214. In examples, the variable dimension of the suction inlets 216 can be between 0.5 and 10 mm.

As discussed herein, the first side port 204 and the second side port 206, and the fluid channel 208 and the suction channel 214 can be combined into a single side port and a single channel (e.g., the channel 120, FIG. 1). In examples, the fluid via the fluid source 212 or the suction via the suction source 218 can be selectively provided to the channel 120. Or, in the examples where the side ports and the channels are separate, as shown in FIG. 2, the fluid and the suction can be provided simultaneously.

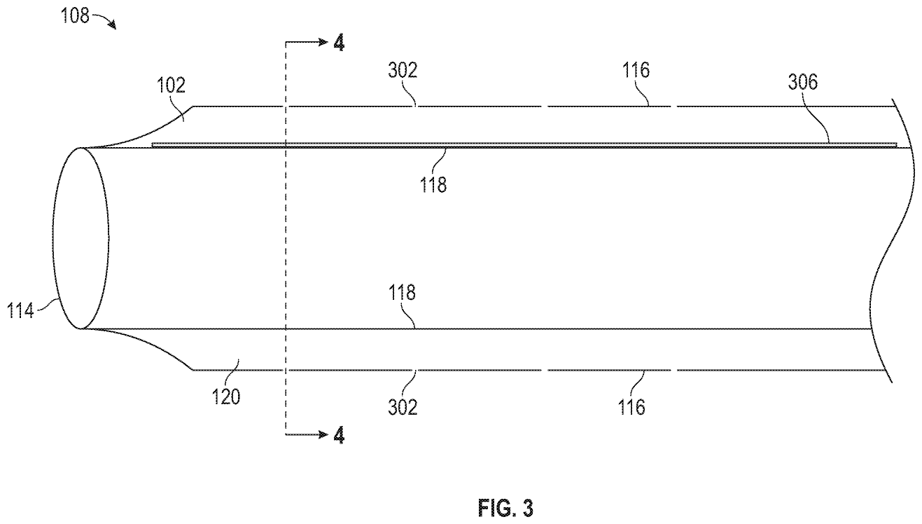

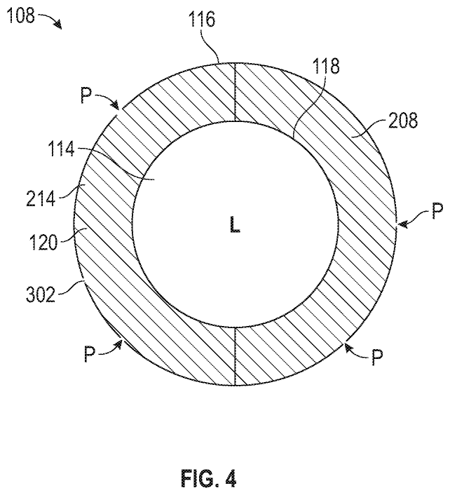

FIG. 3 and FIG. 4 will be discussed together. FIG. 3 illustrates a portion of an example of an esophagus intubation device 100. FIG. 4 illustrates a cross-sectional view of an example of the esophagus intubation device 100 from FIG. 3 taken along indicator line 4-4.

As discussed herein, the outer wall 116 can surround the lumen 114 and can define part of the channel 120. The outer wall 116 can include one or more exits 302 (e.g., the fluid exits 210 or the suction inlets 216, both in FIG. 2) fluidically coupled to the channel 120. In examples, the channel 120 can also be implemented as separate channels, such as the fluid channel 208 and the suction channel 214, as shown in FIG. 2. The one or more exits 302 can direct a fluid to one or more portions of the esophagus. For example, a fluid source (e.g., the fluid source 212, FIG. 2) can be configured to provide the fluid to the channel 120, and one or more exits 302 can introduce the fluid to the esophagus of the patient. The one or more exits 302 can also be configured to draw debris from the one or more portions of the esophagus when a suction source (e.g., the suction source 218, FIG. 2) is connected to the channel 120. For example, the one or more exits 302 can be configured to draw tissue, blood, saliva, any other excess liquid or fluid, any combination thereof, or the like from the esophagus during the medical procedure.

As shown in FIG. 3 and FIG. 4, the channel 120 can be a coaxial channel surrounding the lumen 114. The coaxial configuration of the channel 120 can also be configured to selectively provide a suction source to draw debris from the fluid channel or provide the fluid to the fluid channel. The one or more exits 302 can be dispersed around the outer wall 116 to fluidly connect the coaxially configured channel 120 and the esophagus of the patient. In the coaxial configuration, the channel 120 can distribute the fluid around a circumference of the outer wall 116 and to the esophagus via the one or more exits 302.

In examples, in the coaxial configuration, the channel 120 can include the fluid channel 208 and the suction channel 214 (both in FIG. 2) so that the channel 120 can simultaneously provide fluid to the esophagus and suction to draw debris from the esophagus. For example, the fluid channel and the suction channel can each extend within the channel 120. The fluid channel and the suction channel can each extend in a helical pattern within the channel 120 so that each of the fluid channel and the suction channel extend circumferentially around the channel 120. In other examples, the channel 120 can be split such that the fluid channel takes up a portion of the channel 120 and the suction channel takes up another portion of the channel 120.

As also shown, a cooling channel 306 can be disposed adjacent the inner lumen 114 (e.g., within the channel 120 as shown in FIG. 3). The cooling channel 306 can extend from the proximal end 110 of the elongated tube 108 to the distal end 112 of the elongated tube 108. The cooling channel 306 can be configured to receive a cooling fluid to transfer thermal energy from the cooling fluid to the lumen 114. The cooling channel 306 can limit or inhibit thermal energy buildup within the lumen 114. Limiting the thermal energy buildup within the lumen 114 can help reduce the likelihood of the medical instruments within the lumen 114 overheating and can maintain a safe temperature within the esophagus.

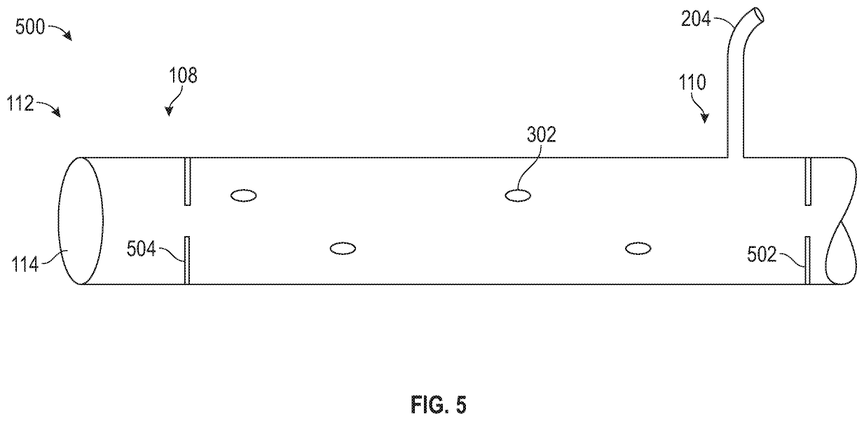

FIG. 5 illustrates a portion of an example of an esophagus intubation device 500. The esophagus intubation device 100 (FIG. 1) can be implemented similar to (e.g., structured similar to) the esophagus intubation device 500. The esophagus intubation device 500 can include a proximal sealing member 502 and a distal sealing member 504 disposed within the lumen 114. The proximal sealing member 502 and the distal sealing member 504 can be configured to create a seal between the outer wall 116 and the intubation probe 102 (FIG. 1). The proximal sealing member 502 and the distal sealing member 504 can be elastic membranes disposed within the lumen 114 and configured to expand and surround the intubation probe 102 to create a seal and prevent leakage of the fluid or debris within the lumen 114 around the intubation probe 102. In examples, the proximal sealing member 502 and the distal sealing member 504 can include inflatable balloons disposed within the lumen 114 and configured to expand and surround the intubation probe 102 to create a seal and prevent leakage of the fluid or debris within the lumen 114 around the intubation probe 102.

The side port (e.g., the first side port 204 or the second side port 206) can extend into the lumen 114 between the proximal sealing member 502 and the distal sealing member 504. The side port extending between the proximal sealing member 502 and the distal sealing member 504 can provide fluid or a suction force to a portion of the lumen 114 between the proximal sealing member 502 and the distal sealing member 504. The proximal sealing member 502 and the distal sealing member 504 can increase the predictability of an amount of fluid dispensed from the one or more exits 302 or the amount of suction provided to the one or more exits 302. Though, only one side port is shown in FIG. 5, one or more additional side ports can also be included in the esophagus intubation device 500.

FIG. 6 illustrates a distal section of an example of an esophagus intubation device 600. The esophagus intubation device 100 (FIG. 1) and the esophagus intubation device 500 (FIG. 5) can be implemented similar to (e.g., structured similar to) the esophagus intubation device 600. The elongated tube 108 can be operable between a collapsed configuration 602 for insertion into the esophagus of a patient and an expanded configuration 604. In the expanded configuration 604, the elongated tube 108 can define the lumen 614, and in the collapsed configuration 602, the lumen 614 can be at least partially collapsed. The elongated tube 108 can passively or actively move between the collapsed configuration 602 and the expanded configuration 604.

In examples, a balloon can be disposed within the elongated tube 108. The balloon can be inflatable to move the elongated tube 108 from the collapsed configuration 602 to the expanded configuration 604 and deflatable to allow the elongated tube 108 to collapse from the expanded configuration 604 to the collapsed configuration 602. The elongated tube 108 can include a pressure-sensitive material configured to move the elongated tube 108 toward the collapsed configuration 602 when exposed to a first pressure and move the elongated tube 108 toward the expanded configuration 604 when exposed to a second pressure. The second pressure can be more than the first pressure.

The elongated tube 108 can also include a time-sensitive component configured to move from the collapsed configuration 602 toward the expanded configuration 604 a set time after being exposed to air, light, fluid, or the like. For example, the elongated tube 108 can be configured to move from the collapsed configuration 602 to the expanded configuration 604 as the elongated tube 108 is removed from packaging. The elongated tube 108 can have a set time it takes for the elongated tube 108 to move from the collapsed configuration 602 to the expanded configuration 604 after the elongated tube 108 is removed from the packaging.

In examples, the elongated tube 108 can include a smart material, e.g., nitinol, or another shape-forming material to move between the collapsed configuration 602 and the expanded configuration 604. For example, the elongated tube 108 can be configured to move from the collapsed configuration 602 toward the expanded configuration 604 when exposed to a temperature, light, or the like, and can move back toward the collapsed configuration 602 from the expanded configuration 604 when exposed to another temperature, light, or the like. For example, a warm temperature can be used to move the elongated tube 108 from the collapsed configuration 602 to the expanded configuration 604, and a cold temperature can be used to move the elongated tube 108 from the expanded configuration 604 to the collapsed configuration 602. In yet another example, the elongated tube 108 can be configured to passively expand over time to move from the collapsed configuration 602 toward the expanded configuration 604 after a set amount of time. The set of amounts of time can permit a clinician to insert the elongated tube 108 within the patient before the elongated tube 108 expands from the collapsed configuration 602 toward the expanded configuration 604.

FIG. 7 illustrates a portion of an example of the bite block 700. In FIG. 7, the bite block 104 can be modular. The bite block 700 can include a first size block 702, a second size block 704, and a third size block 706. The first size block 702, the second size block 704, and the third size block 706 can be configured to be nested within one another, as shown in FIG. 7. The first size block 702 can be removably coupled to the elongated tube 108. The second size block 704 can be removably coupled to the first size block 702. The third size block 706 can be removably coupled to the second size block 704.

The clinician can begin the medical procedure with the first size block 702 coupled to the elongated tube 108. If the first size block 702 is too small, the clinician can couple the second size block 704 to the first size block 702. If the second size block 704 is too small, the clinician can couple the third size block 706 to the second size block 704. In examples, the bite block 104 can include more than three size variation bite blocks. For example, the bite block 700 can be modular and can include four or more different size bite blocks.

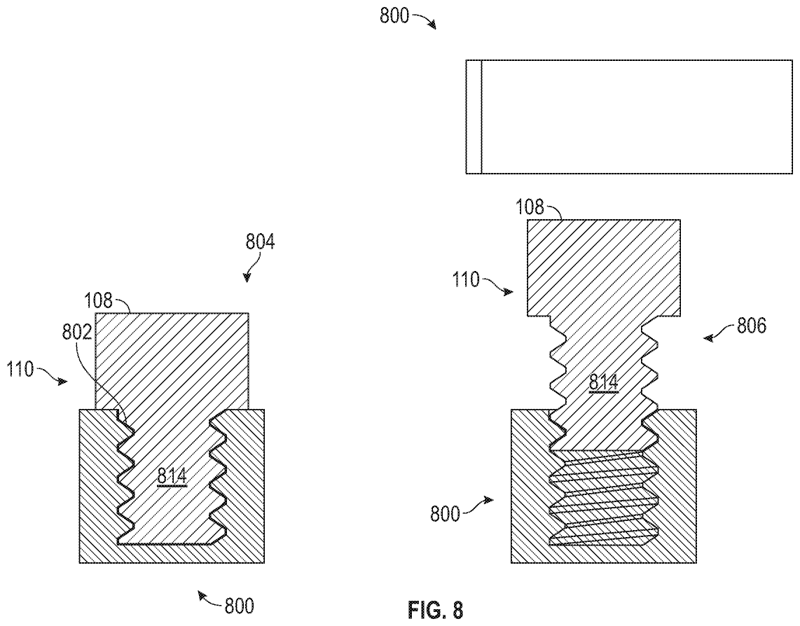

FIG. 8 illustrates an example of a bite block 800. The bite block 800 can include an expandable interface 802. The expandable interface 802 can be configured to operate the bite block 800 between a retracted position 804 and an extended position 806. As shown in FIG. 8, the lumen 814 can extend between the expandable interfaces 802 and can extend into the screen. The bite block 800 can be configured to be used in any position between the retracted position 804 and the extended position 806. As such, the expandable interface 802 can be configured to be a semi-rigid accordion-style interface that can remain at any position between the retracted position 804 and the extended position 806. The expandable interface 802 can enable the bite block 800 to be used with patients of varying mouth sizes or for procedures requiring various mouth openings.

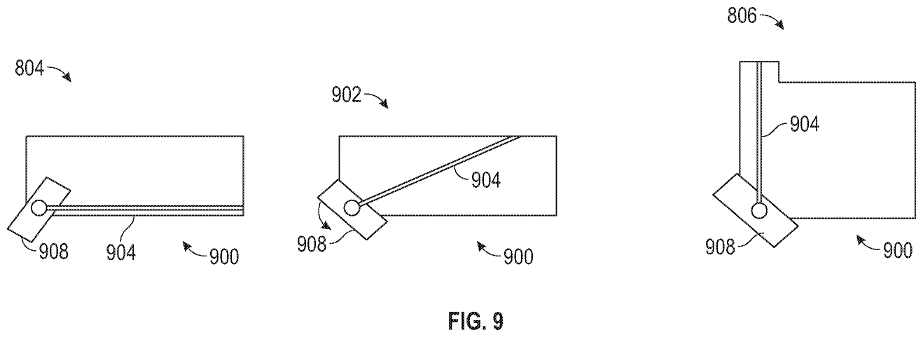

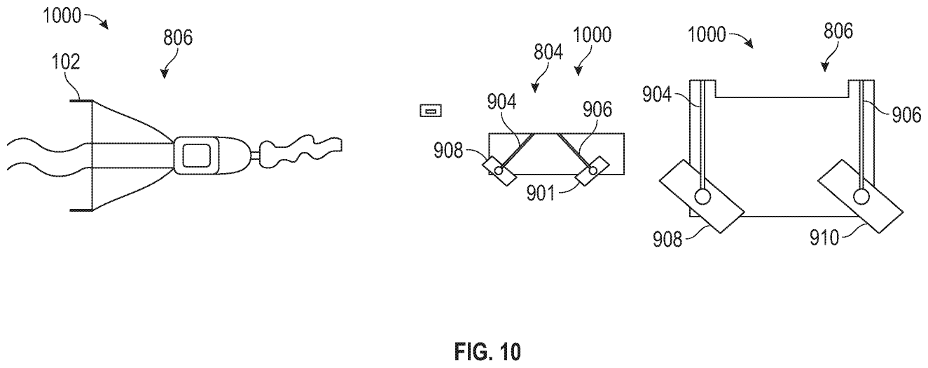

FIG. 9 and FIG. 10 will be discussed together. FIG. 9 illustrates an example of a bite block 900. FIG. 10 illustrates an example of a bite block 1000. The bite block 900 can include a first rigid member 904, a second rigid member 906, a first handle 908, and a second handle 910. The first handle 908 can be coupled to the first rigid member 904. The second handle 910 can be coupled to the second rigid member 906. The first handle 908 and the second handle 910 can be configured to operate (e.g., move, translate, or rotate) the first rigid member 904 and the second rigid member 906, respectively. The first handle 908 and the second handle 910 can, therefore, operate the bite block 900 between the retracted position 804 and the extended position 806.

The bite block 900 can include one of the first handle 908 and the first rigid member 904 (as shown in FIG. 9). The bite block 900 can include the first handle 908 and the first rigid member 904 and the second handle 910 and the second rigid member 906. In examples, the bite block 900 can include just the second handle 910 and the second rigid member 906.

The first handle 908 can be configured to rotate counterclockwise to move the bite block 900 away from the retracted position 804 to an intermediate position 902 or toward the extended position 806. The first handle 908 can be configured to rotate clockwise to move the bite block 900 away from the extended position 806 to the intermediate position 902 or toward the retracted position 804.

As shown in FIG. 10, the second handle 910 can be configured to rotate clockwise to move the bite block 1000 from the retracted position 804 to the intermediate position 902 or toward the extended position 806. The second handle 910 can be configured to rotate counterclockwise to move the bite block 1000 from the extended position 806 to the intermediate position 902 or toward the retracted position 804. In examples, the first handle 908 and the second handle 910 can be configured to hold any position between the furthest most counterclockwise position and the furthest most clockwise position, to hold the bite block 1000 in any position between the retracted position 804, the intermediate position 902, and the extended position 806.

In examples, to lock the position of the first handle 908 or the second handle 910, a lock interface can be included. The lock interface can be integral to the first handle 908 or the second handle 910 or can be configured to be coupled to the first handle 908 or the second handle 910. For example, the lock interface can be configured to lock the position of the first handle 908 or the second handle 910 when the first handle 908 or the second handle 910 are pushed in towards the screen or pulled out away from the screen.

The bite blocks shown in FIGS. 7-10 are just example bite blocks that can be used during medical procedures. In examples, the bite block can be configured to be inserted within a mouth of a patient when it is in a “low profile” or “relaxed” state and can then be expanded within the mouth of the patient. Any of the interfaces, members, handles, or the like shown herein can be used to manipulate the bite block from the “low profile” state to an expanded state. In examples, the bite block can be configured to be moved from the “low profile” state to an expanded state using external tools, e.g., reverse pliers, pliers, forceps, or the like. Once the bite block is appropriately expanded, the bite block can be configured to receive a rigid member set to the expanded configuration to maintain or hold the bite block in the expanded position. In examples, the insert can be a separate component that the user inserts to inhibit or prevent the bite block from moving from the expanded state to the “low profile” state. In examples, the locking mechanism can be integral with the bite block, e.g., the first rigid member, the second rigid member, or the like.

In examples, the bite block can be configured to be moved from the “low profile” state to an intermediate state or the expanded state before being inserted into the mouth of the patient. In other examples, the bite block can be configured to be adjusted throughout the medical procedure based on the clearance requirements of the medical procedure.

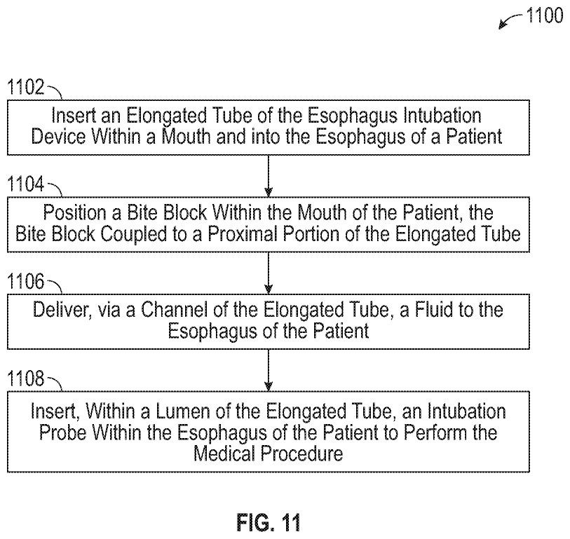

FIG. 11 illustrates an example of a method 1100. The method 1100 can be an example of using an esophagus intubation device (e.g., the esophagus intubation device 100, FIG. 1). The method 1100 can optionally include operations including at least one of operations 1102 through 1108.

At operation 1102, the method 1100 can optionally include inserting an elongated tube (e.g., the elongated tube 108, FIG. 1) of the esophagus intubation device within a mouth and into the esophagus of a patient. As discussed herein, the esophagus intubation device can be operable between a collapsed configuration and an expanded configuration. The esophagus intubation device can be insertable through the mouth and into the esophagus of the patient while in the collapsed configuration. In other examples, the esophagus intubation device can be insertable through the mouth and into the esophagus of the patient while in the expanded configuration.

At operation 1104, the method 1100 can optionally include positioning a bite block (e.g., the bite block 104, FIG. 1) within the mouth of the patient. As discussed herein, the bite block can be coupled to a proximal portion of the elongated tube. The bite block can also be adjustable such as to fit in mouths of varying sizes or can be used with various medical procedures requiring different access to the mouth or the esophagus of the patient. In examples, the bite block adjustments can be made pre-operatively or intraoperatively.

At operation 1106, the method 1100 can optionally include delivering, via a channel (e.g., the channel 120, FIG. 1 or the fluid channel 208, FIG. 2) of the elongated tube, a fluid to the esophagus of the patient. The fluid can include one or more of saline, water, lidocaine, glucose, any biocompatible lubricant, combinations thereof, or the like. The fluid can be dispensed pre-operatively, intraoperatively, or post-operatively.

As discussed herein, the esophagus intubation device can operate between a collapsed configuration and an expanded configuration. In its expanded configuration, the elongated tube can define a lumen. The lumen can be at least partially collapsed in the collapsed state. The elongated tube can move between these configurations either passively or actively. Several mechanisms can facilitate this transition: a balloon within the tube can inflate to expand it and deflate to collapse it; the tube can include pressure-sensitive material that collapses under lower pressure and expands under higher pressure; a time-sensitive component can cause expansion after exposure to air, light, or fluid; or the tube can passively expand over time after insertion. These features can allow a clinician to insert the device while collapsed and have it expand once in place.

In examples, the elongated tube can be configured to move from the collapsed configuration to the expanded configuration at any time between operations 1102 and 1106. For example, the elongated tube can be configured to operate from the collapsed configuration to the expanded configuration after the elongated tube is inserted into the esophagus of the patient. In other examples, the elongated tube can be configured to move from the collapsed configuration to the expanded configuration after fluids are introduced to the esophagus. In examples, the fluids can initiate the expansion of the elongated tube from the collapsed configuration toward the expanded configuration.

At operation 1108, the method 1100 can optionally include inserting, within a lumen (e.g., the lumen 814, FIG. 1) of the elongated tube, an intubation probe (e.g., the intubation probe 102, FIG. 1) within the esophagus of the patient to perform the medical procedure.

The present disclosure relates to an intubation device designed to address challenges in esophageal intubation procedures. Esophageal intubation can be challenging due to anatomical factors, patient-specific variations, and the high skill level required, with traditional verification methods like auscultation often proving unreliable. The device can include a bite block that keeps the mouth of the patient open during procedures. The bite block can also be configured to guide the tongue throughout the medical procedure. The device can also include an elongated tube extending from the bite block. A lumen that can guide intubation probes to the esophagus can extend through the elongated tube. The elongated tube can also include one or more channels for delivering fluids or providing suction.

The device can operate between collapsed and expanded configurations, deliver various fluids such as saline, lidocaine, and lubricants to the esophagus, remove debris through suction inlets, guide various types of intubation probes, and improve predictability and ease of insertion. The bite block can be modular or adjustable to accommodate different mouth sizes, while the elongated tube includes inner and outer walls with channels between them. Fluid exits and suction inlets can be positioned along the outer wall, and the device can include cooling channels to prevent overheating and sealing members to create a seal between the outer wall and the intubation probe.

Users can insert the elongated tube into the mouth and esophagus, position the bite block within the patient's mouth, deliver fluid via the channel to the esophagus, and insert an intubation probe through the lumen to perform medical procedures. The device of the present disclosure aims to simplify esophageal intubation, improve accuracy, and enhance patient safety during medical procedures.

The following, non-limiting examples, detail certain aspects of the present subject matter to solve the challenges and provide the benefits discussed herein, among others.

Example 1 is a device comprising: a bite block configured to be inserted into a mouth of a patient to maintain the mouth open during a medical procedure; and an elongated tube extending from the bite block, the elongated tube operable between a collapsed configuration for insertion through the mouth into a pharynx and into an esophagus of a patient and an expanded configuration, in the expanded configuration, the elongated tube includes: a lumen extending from a proximal end of the elongated tube adjacent the bite block to a distal end of the elongated tube, the lumen configured to guide an intubation probe to the esophagus of the patient via the distal end of the elongated tube; and an outer wall surrounding the lumen and defining part of a fluid channel, the outer wall including one or more exits fluidically coupled to the fluid channel to direct a fluid to anatomy of the patient or to draw debris from the anatomy of the patient.

In Example 2, the subject matter of Example 1 optionally includes wherein the anatomy of the patient includes one or more of the mouth, a pharynx, or the esophagus.

In Example 3, the subject matter of any one or more of Examples 1-2 optionally include wherein the elongated tube includes an inner wall, the fluid channel being located between the inner wall and the outer wall, and the lumen being located within the inner wall.

In Example 4, the subject matter of any one or more of Examples 1-3 optionally include wherein the bite block comprises: an aperture configured to permit the patient to breathe through their mouth while the device is inserted.

In Example 5, the subject matter of any one or more of Examples 1-4 optionally include wherein the elongated tube comprises a side port fluidically connected to the fluid channel to selectively provide the fluid to the fluid channel or provide a suction source to draw debris from the fluid channel.

In Example 6, the subject matter of any one or more of Examples 1-5 optionally include wherein the fluid channel includes a coaxial channel surrounding the lumen, the coaxial channel configured to selectively provide a suction source to draw debris from the fluid channel or provide the fluid to the fluid channel.

In Example 7, the subject matter of any one or more of Examples 1-6 optionally include wherein the fluid channel comprises: a fluid supply channel to fluidically connect a fluid source and the one or more exits to provide the fluid to one or more portions of the anatomy of the patient; and a suction channel to fluidically connect a suction source and the one or more exits to provide suction to draw debris from one or more portions of the anatomy of the patient.

In Example 8, the subject matter of Example 7 optionally includes wherein the one or more exits comprises: a fluid supply exit fluidically connected to the fluid source via the fluid supply channel, the fluid supply exit configured to direct the fluid from the fluid source toward one or more portions of the anatomy of the patient; and a debris suction exit fluidically connected to the suction source via the suction channel, the debris suction exit configured to draw debris from one or more portions of the anatomy of the patient into the suction channel.

In Example 9, the subject matter of Example 8 optionally includes wherein the fluid supply exit comprises: a first fluid supply exit circumferentially spaced around the elongated tube to dispense the fluid at a first portion of the anatomy of the patient; and a second fluid supply exit axially spaced from the first fluid supply exit and circumferentially spaced around the elongated tube to dispense the fluid at a second portion of the anatomy of the patient.

In Example 10, the subject matter of any one or more of Examples 1-9 optionally include wherein the fluid includes one or more of water, saline, lubricant, a cooling fluid, or anesthetic.

In Example 11, the subject matter of any one or more of Examples 1-10 optionally include wherein the outer wall defines the lumen and the elongated tube further includes a distal sealing member and a proximal sealing member, wherein the proximal sealing member and distal sealing member are configured to create a seal between the outer wall and an intubation probe, wherein the fluid channel is defined by an area between the outer wall and the intubation probe.

In Example 12, the subject matter of Example 11 optionally includes wherein the distal sealing member and the proximal sealing member are elastic membranes within the lumen and configured to expand and surround the intubation probe to create a seal and prevent leakage of the fluid or debris within the lumen around the intubation probe and to provide suction around the intubation probe.

In Example 13, the subject matter of any one or more of Examples 11-12 optionally include wherein the distal sealing member and the proximal sealing member are inflatable balloons disposed within the lumen and configured to expand and surround the intubation probe to create a seal and prevent leakage of the fluid or debris within the lumen around the intubation probe and provide suction around the intubation probe.

In Example 14, the subject matter of any one or more of Examples 1-13 optionally include wherein the bite block includes an expansion joint to expand or retract the bite block and accommodate patients with different-sized mouths.

In Example 15, the subject matter of any one or more of Examples 1-14 optionally include wherein the elongated tube comprises: a cooling channel extending from the proximal end of the elongated tube adjacent to the bite block to the distal end of the elongated tube adjacent to the lumen, the cooling channel configured to receive a cooling fluid to transfer thermal energy from the cooling fluid to the lumen.

In Example 16, the subject matter of any one or more of Examples 1-15 optionally include wherein the elongated tube comprises: a balloon disposed within the elongated tube and inflatable to move the elongated tube to the expanded configuration and deflatable to move the elongated tube to the collapsed configuration.

In Example 17, the subject matter of any one or more of Examples 1-16 optionally include wherein the elongated tube includes pressure-sensitive material configured to move the elongated tube to the collapsed configuration when exposed to a first temperature and move the elongated tube to the expanded configuration when exposed to a second temperature, wherein the second temperature is more than the first temperature.

In Example 18, the subject matter of any one or more of Examples 1-17 optionally include wherein the elongated tube includes a self-expanding material configured to expand after a set amount of time after being removed from packaging.

In Example 19, the subject matter of Example 18 optionally includes wherein the elongated tube is configured to passively expand over time to move from the collapsed configuration to the expanded configuration after the elongated tube is inserted into the patient.

Example 20 is a method of using an esophagus intubation device, the method comprising: inserting an elongated tube of the esophagus intubation device within a mouth into a pharynx and into an esophagus of a patient; positioning a bite block within the mouth of the patient, the bite block coupled to a proximal portion of the elongated tube; delivering, via a channel of the elongated tube, a fluid to the mouth, the pharynx, or the esophagus of the patient; and inserting, within a lumen of the elongated tube, an intubation probe within the esophagus of the patient to perform a medical procedure.

In Example 21, the subject matter of Example 20 optionally includes removing, via the channel of the elongated tube, debris within the mouth, pharynx, or esophagus.

Example 22 is a method of using an esophagus intubation device, the method comprising: positioning a bite block within a mouth of a patient; expanding the bite block from a retracted position to an expanded position; inserting an elongated tube of an esophagus intubation device through the bite block and within the mouth into a pharynx and into an esophagus of a patient; coupling the elongated tube to the bite block; delivering, via a channel of the elongated tube, a fluid to the mouth, pharynx, or esophagus of the patient; and inserting, within a lumen of the elongated tube, an intubation probe within the esophagus of the patient to perform a medical procedure.

In Example 23, the subject matter of Example 22 optionally includes removing, via the channel of the elongated tube, debris within the mouth, pharynx, or esophagus.

Example 24 is a device comprising: a bite block configured to be inserted into a mouth of a patient to maintain the mouth open during a medical procedure; and an elongated tube extending from the bite block, the elongated tube operable between a collapsed configuration for insertion through the mouth into a pharynx and into an esophagus of a patient and an expanded configuration, in the expanded configuration, the elongated tube includes: a lumen extending from a proximal end of the elongated tube adjacent the bite block to a distal end of the elongated tube, the lumen configured to guide an intubation probe to the esophagus of the patient via the distal end of the elongated tube; and an outer wall surrounding the lumen and defining part of a fluid channel, the outer wall including one or more exits fluidically coupled to the fluid channel to direct a fluid to one or more of the mouth, the pharynx, or the esophagus of the patient or to draw debris from one or more of the mouth, the pharynx, or the esophagus of the patient.

In Example 25, the subject matter of Example 24 optionally includes wherein the elongated tube includes an inner wall, the fluid channel being located between the inner wall and the outer wall, and the lumen being located within the inner wall.

In Example 26, the subject matter of any one or more of Examples 24-25 optionally include wherein the bite block comprises: an aperture configured to permit the patient to breathe through their mouth while the device is inserted.

In Example 27, the subject matter of any one or more of Examples 24-26 optionally include wherein the elongated tube comprises a side port fluidically connected to the fluid channel to selectively provide the fluid to the fluid channel or provide a suction source to draw debris from the fluid channel.

In Example 28, the subject matter of any one or more of Examples 24-27 optionally include wherein the fluid channel includes a coaxial channel surrounding the lumen, the coaxial channel configured to selectively provide a suction source to draw debris from the fluid channel or provide the fluid to the fluid channel.

In Example 29, the subject matter of any one or more of Examples 24-28 optionally include wherein the fluid channel comprises: a fluid supply channel to fluidically connect a fluid source and the one or more exits to provide the fluid to one or more portions of the mouth, the pharynx, or the esophagus of the patient; and a suction channel to fluidically connect a suction source and the one or more exits to provide suction to draw debris from one or more portions of the mouth, the pharynx, or the esophagus of the patient; and wherein the one or more exits comprise: a fluid supply exit fluidically connected to the fluid source via the fluid supply channel, the fluid supply exit configured to direct the fluid from the fluid source toward one or more portions of the mouth, the pharynx, or the esophagus of the patient; and a debris suction exit fluidically connected to the suction source via the suction channel, the debris suction exit configured to draw debris from one or more portions of the mouth, the pharynx, or the esophagus of the patient into the suction channel.

In Example 30, the subject matter of Example 29 optionally includes wherein the fluid supply exit comprises: a first fluid supply exit circumferentially spaced around the elongated tube to dispense the fluid at a first portion of the mouth, the pharynx, or the esophagus of the patient; and a second fluid supply exit axially spaced from the first fluid supply exit and circumferentially spaced around the elongated tube to dispense the fluid at a second portion of the mouth, the pharynx, or the esophagus of the patient.

In Example 31, the subject matter of any one or more of Examples 24-30 optionally include wherein the outer wall defines the lumen and the elongated tube further includes a distal sealing member and a proximal sealing member, wherein the proximal sealing member and distal sealing member are configured to create a seal between the outer wall and an intubation probe, wherein the fluid channel is defined by an area between the outer wall and the intubation probe.

In Example 32, the subject matter of Example 31 optionally includes wherein the distal sealing member and the proximal sealing member are one of elastic membranes or inflatable balloons disposed within the lumen and configured to expand and surround the intubation probe to create a seal and prevent leakage of the fluid or debris within the lumen around the intubation probe and to provide suction around the intubation probe.

In Example 33, the subject matter of any one or more of Examples 24-32 optionally include wherein the elongated tube comprises: a cooling channel disposed adjacent to the lumen and extending from the proximal end of the elongated tube to the distal end of the elongated tube, the cooling channel configured to receive a cooling fluid to transfer thermal energy away from the lumen.

In Example 34, the subject matter of any one or more of Examples 24-33 optionally include wherein the elongated tube comprises: a balloon disposed within the elongated tube and inflatable to move the elongated tube to the expanded configuration and deflatable to move the elongated tube to the collapsed configuration.

In Example 35, the subject matter of any one or more of Examples 24-34 optionally include wherein the elongated tube includes a shape-memory material configured to move the elongated tube to the expanded configuration when exposed to a first temperature and move the elongated tube to the collapsed configuration when exposed to a second temperature, wherein the first temperature is greater than the second temperature.

In Example 36, the subject matter of any one or more of Examples 24-35 optionally include wherein the elongated tube includes a self-expanding material configured to expand after a set amount of time after being removed from packaging, and wherein the elongated tube is configured to passively expand over time to move from the collapsed configuration to the expanded configuration after the elongated tube is inserted into the patient.

Example 37 is a device comprising: a bite block configured to be inserted into a mouth of a patient, the bite block operable between a retracted position and an expanded position to accommodate patients with different-sized mouths, the bite block defining a cavity providing access to an esophagus of the patient; and an elongated tube removably coupled to the bite block, the elongated tube operable between a collapsed configuration and an expanded configuration, in the expanded configuration, the elongated tube includes: a lumen extending from a proximal end of the elongated tube to a distal end of the elongated tube, the lumen in fluid communication with the cavity of the bite block and configured to guide an intubation probe through the cavity and into the esophagus of the patient; an outer wall surrounding the lumen; and a fluid supply channel and a suction channel disposed between the outer wall and the lumen, the fluid supply channel fluidically connected to one or more fluid exits extending through the outer wall to deliver a fluid to the esophagus of the patient, the suction channel fluidically connected to one or more suction inlets extending through the outer wall to draw debris from the esophagus of the patient, the fluid supply channel and the suction channel being fluidically isolated from one another.

In Example 38, the subject matter of Example 37 optionally includes wherein the bite block comprises an expandable interface configured to operate the bite block between the retracted position and the expanded position, the expandable interface comprising a semi-rigid accordion-style interface configured to remain at any position between the retracted position and the expanded position.

In Example 39, the subject matter of any one or more of Examples 37-38 optionally include wherein the bite block comprises a first rigid member, a second rigid member, and at least one handle coupled to one of the first rigid member or the second rigid member, the at least one handle configured to move the bite block between the retracted position and the expanded position.

Example 40 is a method of using an esophagus intubation device, the method comprising: positioning a bite block within a mouth of a patient, the bite block coupled to a proximal portion of an elongated tube of the esophagus intubation device; inserting the elongated tube within the mouth into a pharynx and into an esophagus of the patient; delivering, via a channel of the elongated tube, a fluid to the mouth, the pharynx, or the esophagus of the patient; and inserting, within a lumen of the elongated tube, an intubation probe within the esophagus of the patient to perform a medical procedure.

In Example 41, the subject matter of Example 40 optionally includes removing, via the channel of the elongated tube, debris within the mouth, pharynx, or esophagus.

In Example 42, the subject matter of any one or more of Examples 40-41 optionally include expanding the bite block from a retracted position to an expanded position prior to inserting the elongated tube; and coupling the elongated tube to the bite block after inserting the elongated tube through the bite block.

In Example 43, the subject matter of any one or more of Examples 40-42 optionally include expanding the elongated tube from the collapsed configuration to the expanded configuration after the elongated tube is inserted into the esophagus of the patient.

The above detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A device comprising:
a bite block configured to be inserted into a mouth of a patient to maintain the mouth open during a medical procedure; and
an elongated tube extending from the bite block, the elongated tube operable between a collapsed configuration for insertion through the mouth into a pharynx and into an esophagus of a patient and an expanded configuration, in the expanded configuration, the elongated tube including:
a lumen extending from a proximal end of the elongated tube adjacent the bite block to a distal end of the elongated tube, the lumen configured to guide an intubation probe to the esophagus of the patient via the distal end of the elongated tube; and
an outer wall surrounding the lumen and defining part of a fluid channel, the outer wall including one or more exits fluidically coupled to the fluid channel to direct a fluid to one or more of the mouth, the pharynx, or the esophagus of the patient or to draw debris from one or more of the mouth, the pharynx, or the esophagus of the patient.

2. The device of claim 1, wherein the elongated tube includes an inner wall, the fluid channel being located between the inner wall and the outer wall, and the lumen being located within the inner wall.

3. The device of claim 1, wherein the bite block comprises:
an aperture configured to permit the patient to breathe through their mouth while the device is inserted.

4. The device of claim 1, wherein the elongated tube comprises a side port fluidically connected to the fluid channel to selectively provide the fluid to the fluid channel or provide a suction source to draw debris from the fluid channel.

5. The device of claim 1, wherein the fluid channel includes a coaxial channel surrounding the lumen, the coaxial channel configured to selectively provide a suction source to draw debris from the fluid channel or provide the fluid to the fluid channel.

6. The device of claim 1, wherein the fluid channel comprises:
a fluid supply channel to fluidically connect a fluid source and the one or more exits to provide the fluid to one or more portions of the mouth, the pharynx, or the esophagus of the patient; and
a suction channel to fluidically connect a suction source and the one or more exits to provide suction to draw debris from one or more portions of the mouth, the pharynx, or the esophagus of the patient; and
wherein the one or more exits comprise:
a fluid supply exit fluidically connected to the fluid source via the fluid supply channel, the fluid supply exit configured to direct the fluid from the fluid source toward one or more portions of the mouth, the pharynx, or the esophagus of the patient; and
a debris suction exit fluidically connected to the suction source via the suction channel, the debris suction exit configured to draw debris from one or more portions of the mouth, the pharynx, or the esophagus of the patient into the suction channel.

7. The device of claim 6, wherein the fluid supply exit comprises:
a first fluid supply exit circumferentially spaced around the elongated tube to dispense the fluid at a first portion of the mouth, the pharynx, or the esophagus of the patient; and
a second fluid supply exit axially spaced from the first fluid supply exit and circumferentially spaced around the elongated tube to dispense the fluid at a second portion of the mouth, the pharynx, or the esophagus of the patient.

8. The device of claim 1, wherein the outer wall defines the lumen and the elongated tube further includes a distal sealing member and a proximal sealing member, wherein the proximal sealing member and distal sealing member are configured to create a seal between the outer wall and an intubation probe, wherein the fluid channel is defined by an area between the outer wall and the intubation probe.

9. The device of claim 8, wherein the distal sealing member and the proximal sealing member are one of elastic membranes or inflatable balloons disposed within the lumen and configured to expand and surround the intubation probe to create a seal and prevent leakage of the fluid or debris within the lumen around the intubation probe and to provide suction around the intubation probe.

10. The device of claim 1, wherein the elongated tube comprises:
a cooling channel disposed adjacent to the lumen and extending from the proximal end of the elongated tube to the distal end of the elongated tube, the cooling channel configured to receive a cooling fluid to transfer thermal energy away from the lumen.

11. The device of claim 1, wherein the elongated tube comprises:
a balloon disposed within the elongated tube and inflatable to move the elongated tube to the expanded configuration and deflatable to move the elongated tube to the collapsed configuration.

12. The device of claim 1, wherein the elongated tube includes a shape-memory material configured to move the elongated tube to the expanded configuration when exposed to a first temperature and move the elongated tube to the collapsed configuration when exposed to a second temperature, wherein the first temperature is greater than the second temperature.

13. The device of claim 1, wherein the elongated tube includes a self-expanding material configured to expand after a set amount of time after being removed from packaging, and wherein the elongated tube is configured to passively expand over time to move from the collapsed configuration to the expanded configuration after the elongated tube is inserted into the patient.

14. A device comprising:
a bite block configured to be inserted into a mouth of a patient, the bite block operable between a retracted position and an expanded position to accommodate patients with different-sized mouths, the bite block defining a cavity providing access to an esophagus of the patient; and
an elongated tube removably coupled to the bite block, the elongated tube operable between a collapsed configuration and an expanded configuration, in the expanded configuration, the elongated tube includes:
a lumen extending from a proximal end of the elongated tube to a distal end of the elongated tube, the lumen in fluid communication with the cavity of the bite block and configured to guide an intubation probe through the cavity and into the esophagus of the patient;
an outer wall surrounding the lumen; and
a fluid supply channel and a suction channel disposed between the outer wall and the lumen, the fluid supply channel fluidically connected to one or more fluid exits extending through the outer wall to deliver a fluid to the esophagus of the patient, the suction channel fluidically connected to one or more suction inlets extending through the outer wall to draw debris from the esophagus of the patient, the fluid supply channel and the suction channel being fluidically isolated from one another.

15. The device of claim 14, wherein the bite block comprises an expandable interface configured to operate the bite block between the retracted position and the expanded position, the expandable interface comprising a semi-rigid accordion-style interface configured to remain at any position between the retracted position and the expanded position.

16. The device of claim 14, wherein the bite block comprises a first rigid member, a second rigid member, and at least one handle coupled to one of the first rigid member or the second rigid member, the at least one handle configured to move the bite block between the retracted position and the expanded position.

17. A method of using an esophagus intubation device, the method comprising:
positioning a bite block within a mouth of a patient, the bite block configured to couple to a proximal portion of an elongated tube of the esophagus intubation device;
inserting the elongated tube within the mouth into a pharynx and into an esophagus of the patient;
delivering, via a channel of the elongated tube, a fluid to the mouth, the pharynx, or the esophagus of the patient; and
inserting, within a lumen of the elongated tube, an intubation probe within the esophagus of the patient to perform a medical procedure.

18. The method of claim 17, further comprising removing, via the channel of the elongated tube, debris within the mouth, pharynx, or esophagus.

19. The method of claim 17, further comprising:
expanding the bite block from a retracted position to an expanded position prior to inserting the elongated tube; and
coupling the elongated tube to the bite block after inserting the elongated tube through the bite block.

20. The method of claim 17, further comprising expanding the elongated tube from a collapsed configuration to an expanded configuration after the elongated tube is inserted into the esophagus of the patient.
